(19) United States
(12) Patent Application Publication
(10) Pub. No.: US 2016/0202223 A1
(43) Pub. Date: Jul. 14, 2016

(54) DETECTION OF FAULTS WHEN DETERMINING CONCENTRATIONS OF CHEMICAL COMPONENTS IN A DISTILLATION COLUMN

(71) Applicant: L'AIR LIQUIDE,SOCIETE ANONYME POUR L'ETUDE ET L'EXPLOITATION DES PROCEDES GEORGES CLAUDE, Paris (FR)

(72) Inventors: Fouad AMMOURI, Massy (FR); Stephane DUDRET, Paris (FR); Pierre ROUCHON, Meudon (FR)

(73) Assignee: L'Air Liquide, Societe, Anonyme pour I'Etude et I'Exploitation des Procedes Georges Claude, Paris (FR)

(21) Appl. No.: 14/910,917

(22) PCT Filed: Aug. 22, 2013
(86) PCT No.: PCT/FR2013/051957

§ 371 Date: Feb. 8, 2016

(51) Int. Cl. G01N 33/00; F25J 3/04

(52) U.S. Cl. CPC G01N 33/007; F25J 3/04412

(57) ABSTRACT

The invention concerns a method for determining the concentrations of chemical components of a product, in particular air, in a distillation column, said method involving implementing a model for estimating the concentration of the components from measurements carried out by one or a plurality of sensors, said model using an adjustment parameter making it possible to take into consideration operating variations of the column, the method also includes a step which involves detecting the values of said adjustment parameter that are outside a nominal variation range of said parameter in order to diagnose a fault D in one or a plurality of said sensors.

### CROSS REFERENCE TO RELATED APPLICATIONS

This application is a §371 of International PCT Application PCT/FR2013/051957, filed Aug. 22, 2013, which is herein incorporated by reference in its entirety.

### TECHNICAL FIELD OF THE INVENTION

The present invention relates to the detection of failures in a device and to a method for determining the concentrations of chemical components, in particular of air, in a distillation column. It also relates to a corresponding air separation unit.

### BACKGROUND OF THE INVENTION

The term “concentrations” is understood more specifically here and in the remainder of the text to mean “mole fractions”. The term “concentrations” is retained for the purpose of simplification.

The utilization of air separation units, known under the acronym ASU, requires knowledge of the concentrations and/or temperatures inside the distillation columns that form these units.

Sensors, referred to as concentration analyzers, exist on the market that make it possible to measure the concentrations of the chemical components in a given location of a distillation column. To enable correct utilization of the column, it is advisable to check the reliability of measurements made by the sensors.

Many methods have already been proposed for this purpose. They are based for the most part on techniques of data reconciliation between the various measurements carried out by the sensors. Their approach is generally limited to steady-state operating modes or, for the most relevant operating modes, transient-state operating modes, provided that there are then well-identified operating conditions. They often rely on black box models, the realism, the development, the maintainability and the effectiveness of which remain debatable, in particular when they are applied to complex industrial units.

Furthermore, besides their prohibitive cost, the use of concentration analyzers makes it necessary to provide additional pipes for removing samples in the columns.

The addition of pipes and the piercing are not desired since they deteriorate the insulation of the columns. Consequently, the number of concentration analyzers that may be used in a column is limited, thus making the measurement of concentrations all along the column impossible.

It is thus advantageous to estimate, in real time, the concentrations at any location of a distillation column while being based on a limited number of installed sensors, this taking into account, in particular, variations in amount of product treated by the distillation column.

Certain solutions have been developed for this purpose. They rely on models that make it possible to reconstruct, by calculation, the concentration of the components along the column from measurements taken by the sensors. The reliability of the results obtained depends of course on the reliability of the model used, but also on the reliability of the sensors. In this way, the importance of a rapid detection of any anomaly in the operation of the sensors is understood.

Various approaches can be envisaged. In a first approach, a model is incorporated into the process for estimating the concentrations that makes provision for the effects of a failure of the sensors. The model makes specific, fictitious data vary, the value of which data is set at zero under normal operating conditions. An alert is then emitted when the value of the fictitious data moves too far away from zero. Another approach consists in detecting the sudden variations in the concentration values obtained. These methods are, however, onerous in terms of the requirement for calculations.

At the same time, a process is known for estimating concentrations along the column using a model, referred to as a wave model. According to this model, the distillation columns are considered to be continuous beds along which the concentration profiles travel like waves propagating along the gas and liquid flows. This results in concentration profiles that have a general S-shaped appearance along the column. The wave model is parameterized with a parameter, referred to as the shape factor, which adjusts the shape of the wave, in particular the flattening of its appearance.

Generally, the shape factor is considered to be constant in existing wave models. Thus, with this model, the wave travels in the column whilst its shape remains constant. However, this hypothesis is false when the operating conditions change, particularly at a high purity. In order to remedy this defect, corrective approaches exist that link the shape factor to the operating conditions using inferential models or an in-line estimation that will make it possible to adapt the appearance of the curve.

### SUMMARY OF THE INVENTION

Although associated with another approach, a method has furthermore been developed by the applicant that uses a model for estimating the concentrations that also utilizes an adjustment parameter, making it possible to take into account operating variations of the column.

Embodiments of the present invention aim to improve the detection of a failure of a concentration sensor while being based on models of this type.

For this purpose, one embodiment of the present invention is a method for determining the concentrations of chemical components of a product, in particular of air, in a distillation column, in particular a packed distillation column, in which method a model is implemented that makes it possible to estimate the concentration of the components from measurements made by one or more sensors, said model utilizing an adjustment parameter that makes it possible to take into account operating variations of the column, characterized in that it comprises a step in which the values of said adjustment parameter that depart from a nominal variation range of said parameter are detected in order to diagnose a failure of one or more of said sensors.

In one embodiment, the invention is based on the observation that with this type of model, as long as the sensors operate correctly, the adjustment parameter varies in a known manner. On the other hand, if one or more of the sensors is faulty, the adjustment parameter varies erratically since it tends to take into account an operating variation of the column that is not real. In this way, it is sufficient to monitor the variations of the adjustment parameter in order to bring to light the failure. Thus, one of the actual variables of the model for estimating the concentration is utilized in order to determine the presence of an anomaly in the operation of the sensors, which facilitates the process.

According to various features of the invention, which could be taken together or separately:
said method comprises a prior step wherein at least one said nominal variation range of said parameter is established,
said prior step comprises a learning step wherein all or some of the values taken by said adjustment parameter over at least one period of time, referred to as reference values, are recorded,
said prior step comprises a step of determining said nominal variation range from said reference values,
said learning step is carried out with said distillation column,
said prior step comprises a step of fusing data originating from several distillation columns and said step of determining said nominal variation range utilizes said data fusion to determine an overall nominal variation range,
said prior step comprises a step of selecting said nominal variation range from nominal ranges of variations corresponding to all or some of said facilities and/or the overall nominal variation range,
said method comprises a step of recording values taken by said adjustment parameter during an excursion of said adjustment parameter outside of said nominal variation range, referred to as the initial nominal variation range, and a step of modifying the initial nominal variation range to give an expanded range, taking into account values taken by the adjustment parameter outside of said initial nominal variation range, when a verification makes it possible to establish that the failure associated with said excursion is a false positive,
several nominal variation ranges are provided, each range corresponding to given operating conditions of said distillation column and said method comprises a step of determining the operating conditions of the distillation column and a step of selecting the corresponding nominal variation range,
said distillation unit comprises several zones each utilizing one said adjustment parameter and one said nominal variation range is used for each of said zones.

This being so, according to one aspect of the invention, the model used is of the type that makes it possible to estimate the concentration of the components as a function of the time and of the position along the longitudinal axis of the column.

More specifically, it could be a model that utilizes a propagation term in connection with a convection of said components along the column and an axial diffusion term in connection with a diffusion of said components in the column, the adjustment parameter making it possible to weight the effects of the diffusion with respect to the effects of the propagation. Specifically, the adjustment parameter which has a very low value, in particular much lower than 1, makes it possible to weight the diffusion term.

The diffusion term of the model of the invention results from exchanges between the liquid and gaseous phases. Specifically, in a packed distillation column, the upward gas, or vapor, flows are in contact with the downward liquid flows. This is modeled simply by a single vapor flow in contact with a single liquid flow, across a single contact interface. At the interface, the liquid and the gas are concomitant and at any moment satisfy the thermodynamic equilibrium, which imposes the concentrations of the components in the liquid phase and in the gaseous phase. The further away from the interface, the less thermodynamically coupled the fluids are. Consequently, far from the interface, the concentrations are different from the concentrations at the interface. In each phase, diffusion flows toward the interface tend to re-homogenize the concentrations. Owing to taking into account not only the phenomenon of convection of the components along the column, but also of the diffusion thereof, the method of an embodiment of the invention provides a judicious estimation of the concentrations.

Moreover, by expressing the diffusion phenomenon along the same axis as that along which the concentrations vary owing to the propagation term, namely the longitudinal axis of the column, the weight of the calculations to be performed is reduced and the simulation may be carried out in real time.

It could be noted that such a model originates from a microscopic analysis, in particular from a formation of equations of some of the phenomena involving the concentration of the components in an infinitesimal cross section of the column. The diffusion term of the model results in this way, in particular, from microscopic exchanges between the liquid and gaseous phases.

Most commonly, since the columns are oriented vertically, the position along the longitudinal axis of the column will represent the vertical position along the column. Certain embodiments of the invention will however also find their applications in columns having another orientation, in particular a horizontal orientation.

By way of example, the model in accordance with an embodiment of the invention delivers the value of the concentrations along an axis, the origin of which is placed at the end of the column at which the concentration of the least volatile compound is minimal and oriented in the increasing direction of the concentration of said least volatile compound. In other words, for air distillation columns, having a vertical orientation, the least volatile compound is oxygen and the origin is the top of the column, the axis being oriented by following the increasing concentration of oxygen, that is to say from top to bottom.

It should be noted that a packed distillation column is understood to mean distillation columns comprising elements that are in the form of metal sheets defining a network of channels for circulation of the liquid and of the gas passing through the column, said sheets being configured so that said channels are greatly interlinked so as to promote contacting of the liquid phases and gaseous phases circulating in said channels. That being so, the invention applies more broadly to any distillation column technology comprising elements that define such a network of fluid circulation channels.

Advantageously, the model uses two different time scales in order to take into account both the longitudinal circulation and the circulation in a direction normal to the interface of the components in the infinitesimal cross section considered, that is to say along a direction transverse to the longitudinal axis of the column, said circulation along a direction normal to the interface being faster than the longitudinal circulation.

Owing to the use of these two scales, all of the physical phenomena occurring in the column are taken into account in the model while allowing simplifications that reduce the weight of calculations to be performed.

According to this aspect of the invention, said model utilizes, for example, a partial differential equation of convection-diffusion linking a first derivative according to time, a first derivative and a second derivative according to the longitudinal position in the column of a value in connection with the concentration of said components in the column, said adjustment parameter being associated with said second derivative.

Said model also utilizes, for example, an approximate expression of the concentration of said components as a function of an intermediate value resulting from solving said equation.

Said approximate expression is, in particular, a truncated expansion with respect to the adjustment parameter, said truncated expansion comprising a zero-order term, expressing the slow phenomena, and a perturbative, first-order term.

Drawn from said partial differential equation is a profile, according to time and the longitudinal position in said column, of said intermediate value and a profile is determined, according to time and the longitudinal position in said column, of the concentration of the components in the column, by transferring said intermediate value to said approximate expression.

By way of example, the partial differential equation has the following form:

$$f ( X ) \frac{\partial X}{\partial t} = \frac{\partial }{\partial z} [ - LX + Vk ( X ) ] + ε \frac{\partial }{\partial z} [ G ( X ) \frac{\partial X}{\partial z} ]$$

in which:
t represents the time;
z represents the position along the axis of the column oriented from the top to the bottom;
L and V represent the respective flow rates of liquid and gas in the column;
X is a vector representing said intermediate value at the time t, at the position z;
k is a matrix of functions, advantageously non-linear functions, of X expressing the thermodynamic equilibrium between the liquid and gaseous phases of the components;
f and G are matrices of functions of X; and
ε is the adjustment parameter.

Matrices of functions are understood to mean applications of the set [0;1]^{A} of the real-valued vectors in the interval [0;1], of dimension A, where A is the size of the vector X, in the set M(R)_{A×N} of the valued matrices in the set of the real numbers R, having A rows and N columns, N being equal to 1 or A.

According to a preferred embodiment, when the mixture contains M components, the size of the vector X is equal to M−1, given that the sum of the concentrations of the components is equal to 1.

By way of example, in an air separation unit, if the components of interest are oxygen, nitrogen and argon, the size of the vector X is equal to 2. In the case of a simplified binary mixture, for example a mixture of oxygen and nitrogen, the vector X is of size 1 and the partial differential equation is then purely scalar.

Said diffusion term is, for example, a function of the flow rate of liquid and gas in the column. In other words, in the equation given above, the matrix of functions G is parameterized by L and V.

The functions f and G could depend on a vector of parameters σ representing the liquid and gas holdups.

According to one embodiment, σ and/or L and/or V are dependent on the time t and/or on the position z.

Preferably, the method comprises a step of numerical solution of the partial differential equation.

By way of example, this numerical solution uses a technique of finite differences in time and space in order to ensure a rapid calculation and a low calculation complexity. According to one embodiment, the time step used for the numerical solution is between a value of the order of a second and a value of the order of a minute, approximately, and the space step is set at 10 centimeters approximately.

The numerical scheme used for processing the equation may be written in implicit or explicit form. In order to have calculated concentrations that are neither negative nor greater than 1, the use of an implicit method may be preferred, even though it requires more calculations.

According to one embodiment, the column comprises points for supplying and/or drawing off the product and/or all or some of the components of the product. Said model divides said column into several sections, referred to as homogeneous sections, each provided between two neighboring supply and/or draw-off points along the height of the column. Said convection term and/or said diffusion term are adapted to each homogeneous section. It will in particular be possible to adapt the vector of parameters G. Adapting the sets of parameters used to each homogeneous section makes it possible to improve the accuracy of the model.

Said model could also utilize boundary conditions describing the principle of mass conservation between two sections of the column, in particular between two homogeneous sections, and at the ends of said column.

The boundary conditions thus complete the model at the ends of a homogeneous section of the packed column. Two cases arise. The first case is that in which the section is located at one end of the column. In this case, the boundary condition expresses a partial or total recycling of the flow leaving the column in order to obtain vapor at the top end of the column and liquid at the bottom end of the column. The second case is that in which the section is connected to another section. In this case, the boundary condition expresses a withdrawal or an injection of liquid and/or of gas between the two adjacent sections.

This being so, said method could additionally comprise the steps of:
measuring the concentration of at least one of said components in at least one location of the column (14, 26); and
adjusting the model with the aid of the adjustment parameter determined from the concentration measured.

More specifically, according to said method, it will be possible, in particular iteratively:
to estimate the concentration of said component at said location of the column where the measurement took place with the aid of said model with a first value of said adjustment parameter,
to establish an error between the estimated value and the measured value of the concentration,
to establish a second value of the adjustment parameter as a function of said error,
to replace the first value of the adjustment parameter by the second value in said model.

Thus, the comparison of the concentrations measured at determined locations of the column, for example with a concentration analyzer, and concentrations determined at the same locations using the model of the invention provides estimation errors which are then used to adjust the model. The concentration measured could in particular be a concentration at the top and/or bottom of the column, a site of high purity of one of the components of the mixture.

The invention also relates to a device for determining the concentrations of chemical components of a product, in particular of air, in a distillation column, in particular a packed distillation column, said device comprising one or more sensors, means for implementing a model that makes it possible to estimate the concentration of the components, from measurements made by the sensor(s), said model utilizing an adjustment parameter that makes it possible to take into account operating variations of the column, characterized in that it additionally comprises means for detecting the values of said adjustment parameter that depart from a nominal variation range of said parameter so as to be able to diagnose a failure of one or more of said sensors. Said device is in particular configured for the implementation of the method described above.

In another embodiment, the invention also relates to an air separation unit comprising at least one air distillation column and a device for determining the concentrations of components of the air in the column as described above.

In another embodiment, the invention also relates to a computer program comprising instructions for the implementation of the method mentioned above, when the program is executed by a processor.

In another embodiment, the invention also relates to a recording medium wherein said program is stored.

### BRIEF DESCRIPTION OF THE DRAWINGS

These and other features, aspects, and advantages of the present invention will become better understood with regard to the following description, claims, and accompanying drawings. It is to be noted, however, that the drawings illustrate only several embodiments of the invention and are therefore not to be considered limiting of the invention's scope as it can admit to other equally effective embodiments.

Exemplary embodiments of the invention will now be described more specifically, but nonlimitingly, with regard to the appended drawings in which:

FIG. 1 is a synoptic diagram illustrating the structure and the operation of an air separation unit according to one embodiment of the invention;

FIG. 2 is a diagram illustrating an infinitesimal section of a column of the separation unit from FIG. 1;

FIG. 3 is a diagram illustrating the phenomena at play in the infinitesimal section from FIG. 2;

FIG. 4 is a diagram illustrating the operation of the method for determining concentrations according to one embodiment of the invention;

FIG. 5 illustrates a first example of a process for detecting a failure of a sensor of an air separation unit according to the method in accordance with the invention, by means of a representation of the value of said adjustment parameter as a function of time;

FIG. 6 illustrates a learning phase of a second example of a process for detecting a failure of a sensor of an air separation unit according to the method in accordance with the invention, by means of a representation of the value of said adjustment parameter as a function of time;

FIG. 7 illustrates a utilization phase of the process from FIG. 6, by means of a representation of the value of said adjustment parameter as a function of time;

FIG. 8 illustrates a learning phase of a third example of a process for detecting a failure of a sensor of an air separation unit according to the method in accordance with the invention, by means of a representation of the value of said adjustment parameter as a function of time;

FIG. 9 illustrates a utilization phase of the process from FIG. 8, according to a first variant, by means of a representation of the value of said adjustment parameter as a function of time;

FIG. 10 illustrates a utilization phase of the process from FIG. 8, according to a second variant, by means of a representation of the value of said adjustment parameter as a function of time;

FIG. 11 illustrates a division of an air separation unit into several zones according to one mode of implemention of the method in accordance with the invention,

FIG. 12 illustrates a learning phase of another mode of implemention of the method in accordance with the invention, by means of a representation of the value of said adjustment parameters, associated with each of the zones of said separation unit from FIG. 11, as a function of time;

FIG. 13 illustrates a utilization phase of said mode of implementation, by means of a representation of the value of said adjustment parameters, associated with each of the zones of said separation unit from FIG. 11, as a function of time, and

FIG. 14 illustrates states of said separation unit from FIG. 11, as a function of time, according to the observations made in connection with FIG. 13.

### DETAILED DESCRIPTION

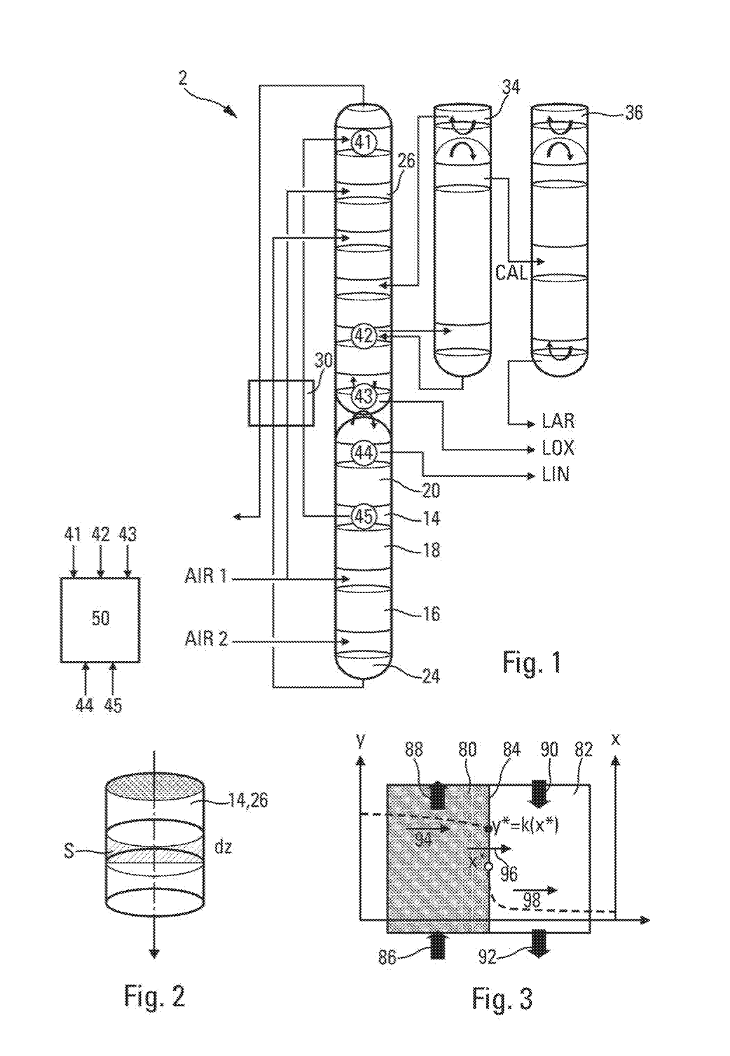

FIG. 1 illustrates a cryogenic air separation unit 2 according to one embodiment of the invention. This unit makes it possible to obtain practically pure oxygen, nitrogen and argon from air, whether this is in liquid or gaseous form.

In a known manner, said separation unit 2 comprises a first packed distillation column 14, referred to as a high-pressure column, and a second packed distillation column 26, located here in the vertical continuity of the high-pressure column 14. It also comprises a third packed distillation column 34, referred to as a crude argon column, and a fourth packed distillation column 36, referred to as a pure argon column.

The high-pressure column 14 comprises several homogeneous sections 16, 18, 20, here three homogeneous sections. The low-pressure column 26 also comprises several homogeneous sections, here five homogeneous sections. The crude argon column here comprises a single homogeneous section. The pure argon column comprises several homogeneous sections, here two homogeneous sections.

At each end of a homogeneous section of one of the columns there takes place either an introduction of air or an introduction of one or some components, resulting from the distillation in one of the other columns, or a drawing-off of one or some components resulting from the distillation in the column in question, this being in the liquid phase and/or in the gaseous phase. In this way a distillation of the type known under the name of reflux distillation is carried out.

Without going further into detail, the high-pressure column 14 is supplied with air in the liquid phase, as illustrated by the arrow associated with the reference AIR 1, and in the gaseous phase, as illustrated by the arrow associated with the reference AIR 2. Supplying with air in the gaseous phase takes place at the bottom of the column and supplying with air in liquid form takes place above the homogeneous section 16 that is found just above the bottom 24 of the high-pressure column 14. Virtually pure liquid nitrogen, denoted by LIN in FIG. 1, is recovered at the top of the high-pressure column 14. Virtually pure liquid oxygen, denoted by LOX, is recovered at the bottom 28 of the low-pressure column 26. Argon-rich liquid, denoted by LAR, is recovered at the bottom of the pure argon column 36.

The separation unit 2 could additionally comprise a heat exchanger 30 at which some of the flows introduced into and/or drawn off from the high- and/or low-pressure columns 14, 26 exchange heat. Said high- and low-pressure columns could furthermore be configured in order to allow a heat exchange between the top of said high-pressure column 14 and the bottom of said low-pressure column 26 in order to respectively enable a liquefaction and a vaporization of the components that are found at this level.

Advantageously, three oxygen concentration analyzers 41, 42, 43 are placed at determined locations of the low-pressure column 26 and two analyzers 44, 45 are placed at determined locations of the high-pressure column 14.

Furthermore, the separation unit 2 comprises a device 50 for determining the concentrations of the chemical components of the air at any location of the high-pressure column 14 and low-pressure column 26. This device 50 comprises, in particular, a processor enabling the utilization of a model describing the variation of the concentrations of the components as a function of time and of the position along each of these columns 14, 26.

Said model uses the measurements made by the analyzers 41, 42, 43, 44, 45, while utilizing an adjustment parameter that makes it possible to take into account the operating variations of the column.

The operation of this device 50 is explained in detail in the remainder of the description, in connection with an embodiment of said model that utilizes a propagation term in connection with a convection of said composants along the column and an axial diffusion term in connection with a diffusion of said components in the column, the adjustment parameter making it possible to weight the effects of the diffusion relative to the effects of the propagation.

In this description, the term column, without any other specification, will denote one of the columns 14, 26 from FIG. 1.

The model used by the device 50 comprises the following partial differential equation (1):

$$f ( X ) \frac{\partial X}{\partial t} = \frac{\partial }{\partial z} [ - LX + Vk ( X ) ] + ε \frac{\partial }{\partial z} [ G ( X ) \frac{\partial X}{\partial z} ] \qquad ( 1 )$$

in which:
t represents the time;
z represents the position along the axis of the column oriented from the top to the bottom;
L and V represent the respective flow rates of liquid and gas in the column;
X is a vector representing an intermediate value linked to the concentration of the components at the time t, at the position z;
k is a matrix of functions expressing the thermodynamic equilibrium between the liquid and gaseous phases of the components;
f and G are matrices of functions of X; and
ε is the adjustment parameter.
The function k could be non-linear. By way of example, it is expressed in the following manner:

$$k ( x ) = \frac{α x}{1 + ( α - 1 ) x}$$

in which α is the relative volatility of the component in question with respect to the compound of row M, the concentration of which is not calculated but deduced from the calculated concentration of the other compounds, as explained above.

The first term of equation (1) represents the propagation term. The second term of equation (1) represents the axial diffusion term. It originates from taking into account a rapid microscopic phenomenon, namely the transverse microscopic diffusion, after simplification.

The microscopic origins of equation 1 will now be described in detail with reference to FIGS. 2 and 3. For the clarity of the description, the mixture is considered to be a binary oxygen/nitrogen mixture, so that equation 1 is scalar, x and y corresponding to the concentration of oxygen, respectively in the liquid phase and in the gaseous phase.

FIG. 2 represents an infinitesimal section S of height dz of the column 14, 26 in which the convection and diffusion phenomena are studied.

In the packed distillation column 14, 26, the upward gas flows are in contact with the downward liquid flows.

With reference to FIG. 3, this physical phenomenon may be modeled simply as a single gas flow 80 in contact with a single liquid flow 82 across a single contact interface 84.

The arrows 86, 88 show the vertical movement from bottom to top of the gas flow 80 and the arrows 90, 92 show the vertical movement from top to bottom of the liquid flow 82, the axis of the abscissae representing the distance at the liquid/gas interface and the axes of the X and Y coordinates representing the concentrations of liquid and of gas respectively.

At the interface 84, the liquid and the gas are concomitant and at thermodynamic equilibrium at any instant, which imposes the concentrations at the interface according to the relationship (2):

y*=k(x*) (2)

in which the asterisk “*” indicates that it is a variable evaluated at the interface 84.

Far from the interface, the fluids are no longer thermodynamically coupled so that the concentrations are different from the concentrations at the interface.

The downward movement of liquid represented by the arrows 90, 92 is described by the relationship (3):

$${σ}_{L} \frac{\partial x}{\partial t} = \frac{\partial ( Lx )}{\partial z} + \frac{{λ}_{L}}{ε} ( {x}^{*} - x ) \qquad ( 3 )$$

in which σ_{L} represents the liquid-phase holdup of the component in question and λ_{L} represents a liquid-phase diffusion coefficient.

Similarly, the upward movement of gas represented by the arrows 86, 88 is described by the relationship (4):

$${σ}_{V} \frac{\partial y}{\partial t} = \frac{\partial ( Vy )}{\partial z} + \frac{{λ}_{V}}{ε} ( {y}^{*} - y ) \qquad ( 4 )$$

in which σ_{V} represents the vapor-phase holdup of the component in question and λ_{V} represents a vapor-phase diffusion coefficient.

Remarkably, said model does not make do with being placed in a slow time scale, in which the radial diffusion phenomenon enabling a mass exchange between the liquid and gaseous phases is disregarded since it is too rapid.

On the contrary, the model of the invention also uses a rapid scale in order to describe this circulation phenomenon represented by the arrows 94, 96, 98.

In each phase, diffusion flows 94, 98 tend to re-homogenize the concentrations. The diffusion then ends up affecting the interface 84 which cannot accumulate or create matter. Thus, a mass exchange flow 96 must cross the interface 84 and thus makes it possible to couple the diffusion flows of each phase. This mass exchange between the 2 phases is expressed by the relationship (5):

$$\frac{{λ}_{V}}{{λ}_{L} ɛ} ( {Y}^{*} - Y ) + \frac{1}{ɛ} ( {X}^{*} - X ) = 0 \qquad ( 5 )$$

The adjustment parameter E is very small, in particular much less than 1. The term λ_{V}/ε can be assimilated into the diffusion coefficient associated with the diffusion flows in the gaseous phase, and the term λ_{L}/ε can be assimilated into the diffusion coefficient associated with the diffusion flows in the liquid phase. The hypothesis according to which the diffusion coefficients are very large is reasonable when the column packing is efficient. With this hypothesis, the system of equations (2) to (5) may be simplified.

In order to carry out this simplification, a technique referred to as the invariant manifold technique is used here, in particular a technique referred to as the center manifold technique. This technique makes it possible to preserve an overall mass balance. It also makes it possible not to make one phase predominant with respect to the other in the structure of the model, in particular from the point of view of liquid/vapor holdups and from the point of view of the thermodynamic equilibrium.

The reduction then leads to equation (1), in which the function G makes it possible to link the operating conditions of the column to the effects of the diffusion.

The function G could be expressed in the following way:

$$G ( X ) = \frac{\frac{{{k}^{′} ( X )}^{2}}{{λ}_{L}} + \frac{{k}^{′} ( X )}{{λ}_{V}}}{{( {σ}_{L} + {σ}_{V} {k}^{′} ( X ) )}^{2}} {( {σ}_{V} L + {σ}_{L} V )}^{2} \qquad ( 6 )$$

in which k′ is the derivative function of the function k. It makes it possible to demonstrate the local effects of L and V on the diffusion.

The function f could be expressed in the following way:

f(X)=σ_{L}+σ_{V}k′(X) (7)

It should be noted that the parameters σ, L and V could depend on the time t and on the position z.

The model also makes it possible to describe the concentrations in each phase.

More specifically, use is made for this of an approximate expression of the concentration of said components as a function of the intermediate value resulting from solving equation (1). Said approximate expression is, for example, a truncated expansion with respect to the adjustment parameter, said truncated expansion comprising a zero-order term, expressing the slow phenomena, and a perturbative, first-order term. This is understood to mean that the zero-order term expresses an operation of the system in which the rapid phenomena are considered to be instantaneous and the perturbative, first-order term takes into account, at least partially, the non-instantaneousness of said rapid phenomena.

The liquid-phase concentration x could be expressed, for example, in the following way:

$$x ( z , t ) = X - {εσ}_{V} \frac{G ( X )}{{σ}_{V} L + {σ}_{L} V} \frac{\partial X}{\partial z} \qquad ( 8 )$$

The gas-phase concentration y could be expressed, for example, in the following way:

$$y ( z , t ) = k ( X ) + {εσ}_{L} \frac{G ( X )}{{σ}_{V} L + {σ}_{L} V} \frac{\partial X}{\partial z} \qquad ( 9 )$$

Thus, in order to estimate the liquid-phase and gaseous-phase concentrations of a component, it will be possible to draw from equation (1) a profile, according to time and the vertical position in the column, of said intermediate value X and then to determine a profile, according to time and the vertical position in said column, of the liquid-phase concentration x and gaseous-phase concentration y of the components in the column, by transferring said intermediate value X to said approximate expression (8) and/or (9).

It is this approach which is implemented here in the device 50.

Besides equation (1), the model comprises boundary conditions describing here the principle of mass conservation between two sections, in particular between two homogeneous sections, of the column and at the ends of said column. More particularly, the effects of the diffusion in equation (1) must be preserved at these boundary locations.

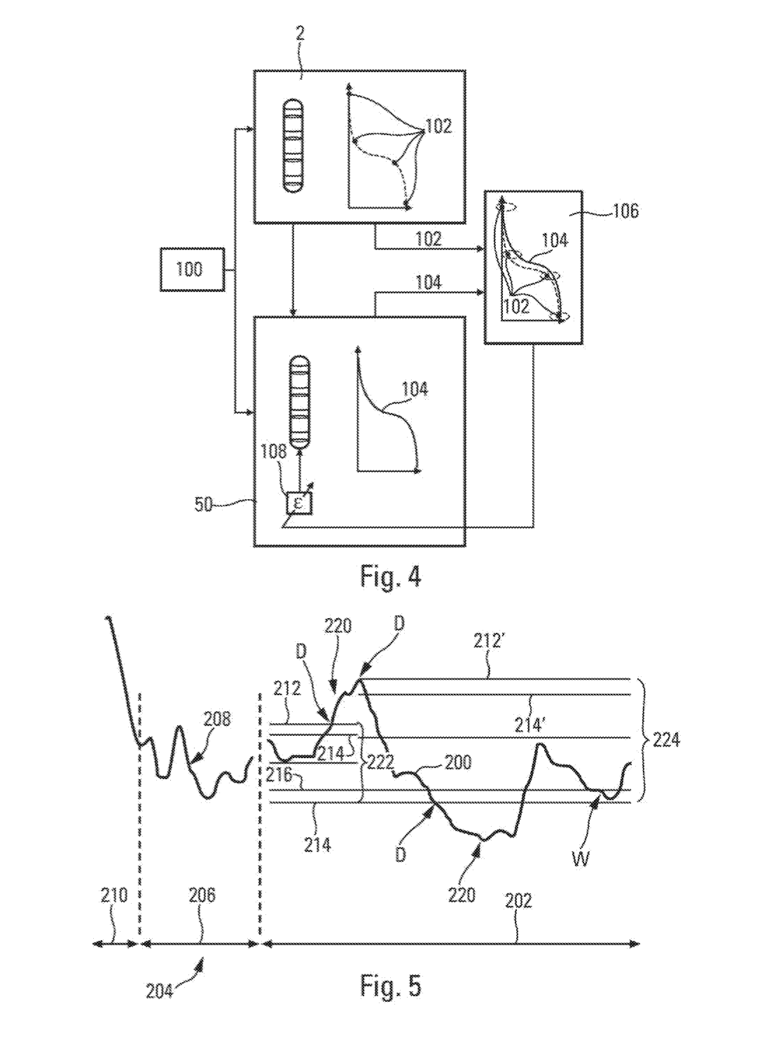

FIG. 4 illustrates the operation of the device 50 for determining the oxygen concentration profile in the air separation unit 2.

Known data 100 are provided to the determining device 50. These are in particular temperatures and/or pressures and/or liquid and/or gas flow rates at determined locations of the separation unit 2.

Moreover, the concentration analyzers 41, 42, 43, 44, 45 provide the determining device 50 with discrete measurements 102 of the oxygen concentration in determined locations of the columns 14, 26. An initial version of the model is thus established. As a variant, arbitrary starting values could also be selected.

Starting from these data, the determining device 50 provided with the model represented by equation (1) iteratively estimates the oxygen concentration profile in the columns 14, 26.

During the first iteration, the adjustment parameter E is set at a certain value. The determining device 50 estimates an oxygen concentration profile 104 using the model which for its part is incorporated with this value of the adjustment parameter.

Next, the determining device 50 compares the concentrations estimated at the determined locations with the discrete measurements and estimation errors (block 106 in FIG. 4) are deduced therefrom which it uses to adapt the adjustment parameter E (block 108 in FIG. 4).

Thus, at the start, during the very first iterations, the concentration profile may be very inaccurate. After a certain time, the parameter E is correctly adjusted. The determining device 50 then provides an accurate concentration profile.

Preferably, each column 14, 26 has its own adjustment parameter E.

During the estimation of the concentration profile, the determining device 50 numerically solves the partial differential equation (1).

For this it uses a technique of finite differences in time and space in order to ensure a rapid calculation of low complexity. The time step selected for the numerical solution is set here at one second approximately and the space step is set at 10 centimeters approximately.

The numerical scheme used for processing the equation is written so that the calculated concentrations are neither negative nor greater than 1, for example with the aid of an implicit scheme.

According to one preferred embodiment, the principle 108 for adapting the adjustment parameter is the following:
if the adjustment parameter E has a correct value, then the mathematical model of equation (1) is realistic. In this case, the estimation errors are zero;
if the estimation errors are not zero, then the mathematical model is not correct. Consequently, it is necessary to change the value of the adjustment parameter E.

Here, the determining device 50 uses an additional equation (10):

$$\frac{d ɛ}{d t} = M \qquad ( 10 )$$

in which M is a function of the estimation errors and optionally of other parameters.

Equation (10) makes it possible to modify the adjustment parameter E continuously in order to keep the estimation errors as low as possible.

The function M may, for example, directly use one or more estimation errors and may take into account other parameters such as the liquid and/or gas flow rates, the pressures, etc.

A simple linear function M that depends only on a single estimation error may be used. It is also possible to use a more complex structure for the function M in order to accelerate the reduction of the estimation errors.

This being so, FIGS. 5 to 10, 12 and 13 illustrate a curve 200 giving the value of the adjustment parameter E as a function of time.

According to the invention, said method for determining the concentrations comprises a step 202, also referred to hereinafter as the utilization phase, in which the values of said adjustment parameter that depart from a nominal variation range of said parameter are detected. In this way a failure D of one or more of said sensors is diagnosed, for example by emitting an alert. Thus, owing to the invention, good use is made of one of the variables already used to obtain an evaluation of the value of the concentrations of the components in order to additionally detect an anomaly of the sensors being used for said evaluation.

Advantageously, said method comprises a prior step 204 in which at least one said nominal variation range of said parameter is established.

Said prior step 204 could comprise a learning step 206 in which all or some of the values taken by said adjustment parameter over at least one period of time, referred to as reference values 208, are recorded.

As is more particularly illustrated in FIG. 5, said prior step could also comprise an initialization step 210 during which the values of the adjustment parameter are ignored in order to avoid taking it into account while it is still too inaccurate, such as the eventuality thereof explained above.

Said prior step 204 could additionally comprise a step of determining said nominal variation range from said reference values 206. It could be a step in which the minimum and maximum values taken by said adjustment parameter are identified. Said determined variation range is here located between a maximum allowable value 212, 212′ and a minimum allowable value 214.

An intermediate range located here between an intermediate upper threshold value 214, 214′ and an intermediate lower threshold value 216 could also be provided. The model in accordance with the invention is then configured in order to emit a warning W when the value of the adjustment parameter departs from said intermediate range.

Following said prior phase, said model is utilized by monitoring the values taken by said adjustment parameter. It will thus be possible to record the values taken by said adjustment parameter during an excursion 220 of said adjustment parameter outside of said nominal variation range. In particular it will be possible to then trigger operations for verifying the sensors.

On this subject, according to one embodiment of the invention, said method could comprise a step of modifying an initial, nominal variation range 222 to give an expanded range 224, taking into account values taken by the adjustment parameter during said excursion 220 when the verification carried out makes it possible to establish that the associated failure is a false positive. A false positive is understood to mean the emission by the model of an alert signifying the presence of an anomaly when the anomaly, after verification, is not real. In other words, all the sensors are in working order.

Here, the maximum allowable value 212 associated with the initial nominal variation range is replaced by the upper allowable value 212′ of the expanded nominal variation range 224. The same is true with the intermediate range for which the intermediate upper threshold value 214 associated with the initial nominal variation range is replaced by the intermediate upper threshold value 214′ associated with the expanded nominal variation range 224.

In other words, in FIG. 5, the excursion 220 that takes place first in time, by higher values, corresponds to a step of updating the nominal variation range of said adjustment parameter, only the excursion 220 that takes place secondly, by lower values, corresponding to the detection of an actual failure D. Furthermore, it will be possible to observe that the warning W emitted afterwards is not followed by the detection of a failure, the value of the adjustment parameter subsequently coming back within the intermediate range.

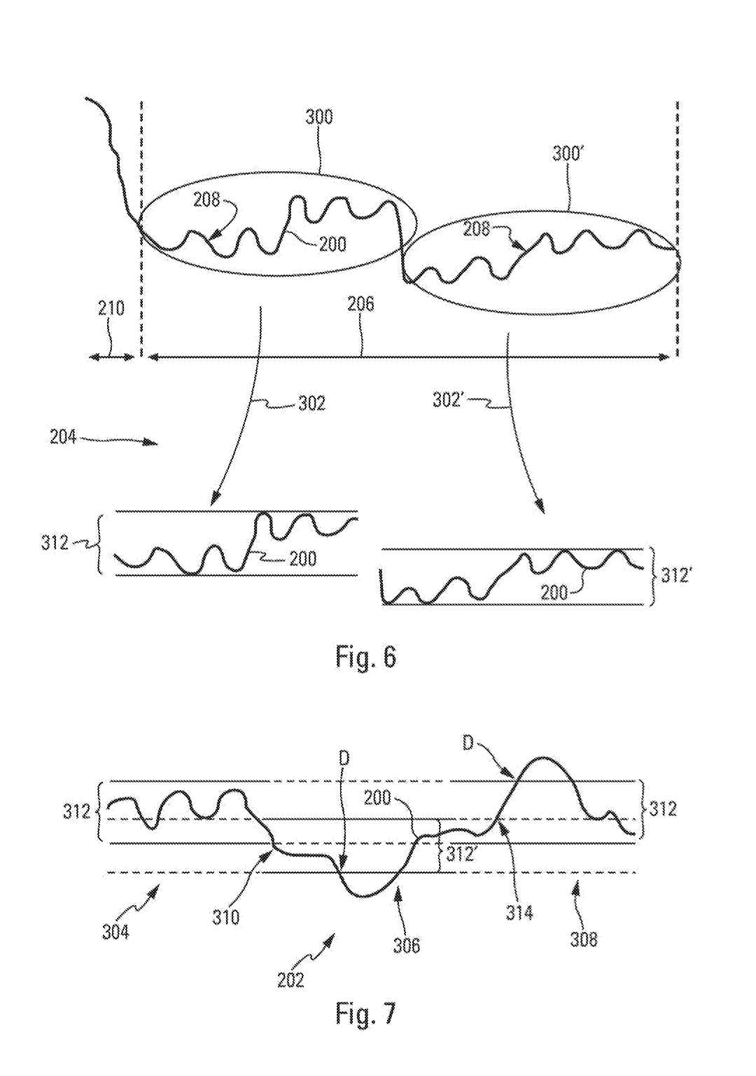

According to one advantageous embodiment illustrated in FIGS. 6 and 7, several nominal variation ranges 312, 312′ are provided, each range corresponding to given operating conditions 300, 300′ of said distillation column.

More specifically, as illustrated in FIG. 6, in the learning phase 206, various operating conditions 300, 300′ are detected and one of said nominal variation ranges 312, 312′ is associated with each of said operating conditions 300, 300′, as symbolized by the arrows 302, 302′ illustrated.

As illustrated in FIG. 7, once in the utilization phase, said method comprises a step of determining the operating conditions of the distillation column and a step of selecting the corresponding nominal variation range 312, 312′, in an iterative manner.

Represented here is a succession of a first utilization phase 304 corresponding to a first nominal variation range 312, followed by a second utilization phase 306 corresponding to a second nominal variation range 312′, followed by a third utilization phase 308 in which the first nominal variation range 312 is found.

It is observed that such an embodiment avoids emitting an alert corresponding to a failure when the value of the adjustment parameter departs, by lower values, from the first nominal variation range 312 at the point 310 since the second operating phase 306 has then been passed into, in which operating phase the value of the adjustment parameter is then found in the corresponding nominal variation range 312′. The same is true at the point 314 although the value of the adjustment parameter this time departs, by higher values, from the second nominal variation range 312′, the first nominal variation range 312 then being applicable. The alerts corresponding to the failures D that are emitted, here during the second 306 and third 308 operating phases, are then more relevant since account is taken of the various operating conditions of the column.

According to a first variant, corresponding to FIGS. 5, 6 and 7 already commented upon, said learning step 206 is carried out with said distillation column only.

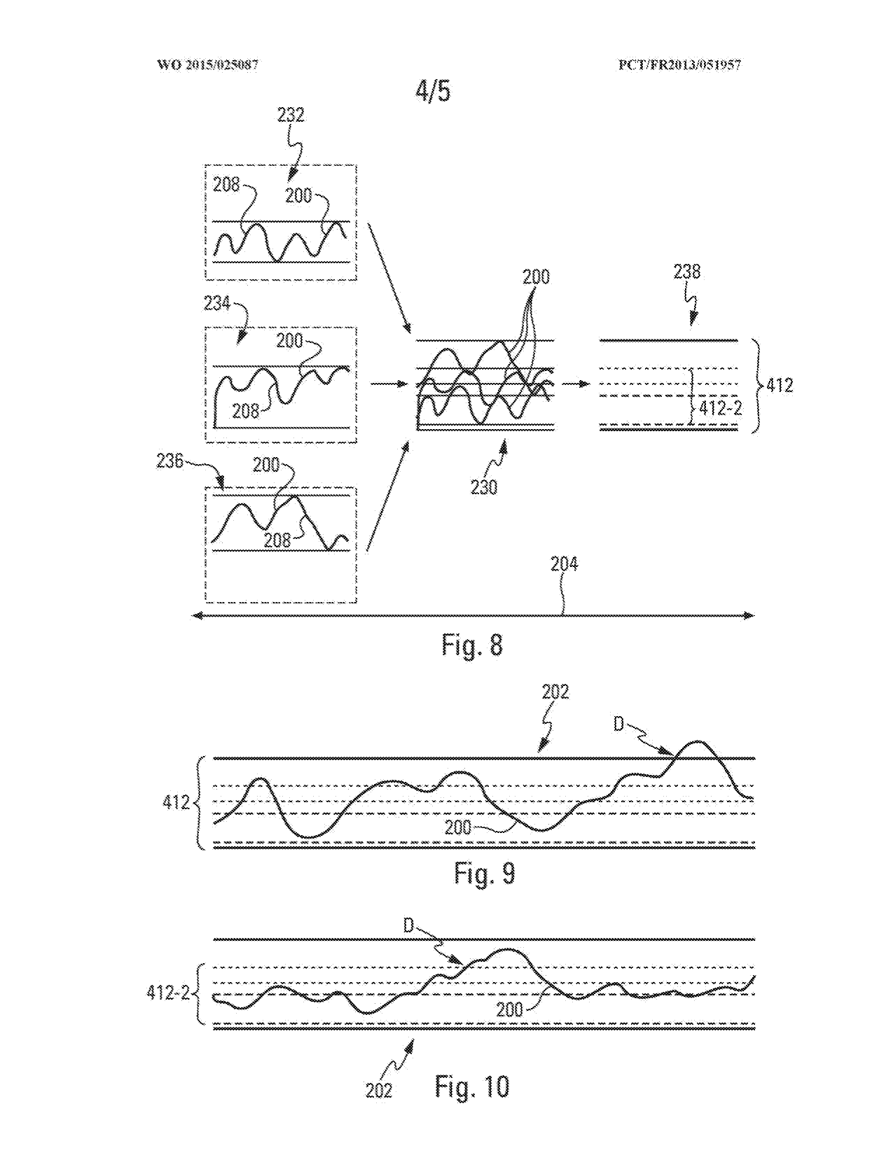

According to another variant, illustrated in FIG. 8, said prior step 204 comprises a step 230 of fusing data 232, 234, 236 originating from several distillation units or columns and said step of determining said nominal variation range, then referred to as the overall nominal variation range, utilizes said data fusion. Said prior step then comprises a step 238 of selecting said nominal variation range from among the nominal variation ranges corresponding to each of said units and the overall nominal variation range 412. Here, only the nominal variation range 412-2 corresponding to the data 234 from the second unit has been identified and illustrated for the sake of ease of representation.

In this way, several possible nominal variation ranges are available, namely, for example, said overall nominal variation range 412, provided as being the broadest, and nominal variation ranges 412-2 corresponding to all or some of the distillation units or columns from which the data originate. In this way it is possible to shorten, or even dispense with the learning phase by using the history from certain facilities, in particular facilities known for close or similar operation.

As illustrated in FIGS. 9 and 10, in the utilization phase one of said nominal variation ranges is then selected in order to utilize said model. In FIG. 9, the overall nominal variation range 412 was selected. In FIG. 10, it is the range 412-2 corresponding to the data from the second utilization. In this way the difference regarding the emission of alerts D is observed.

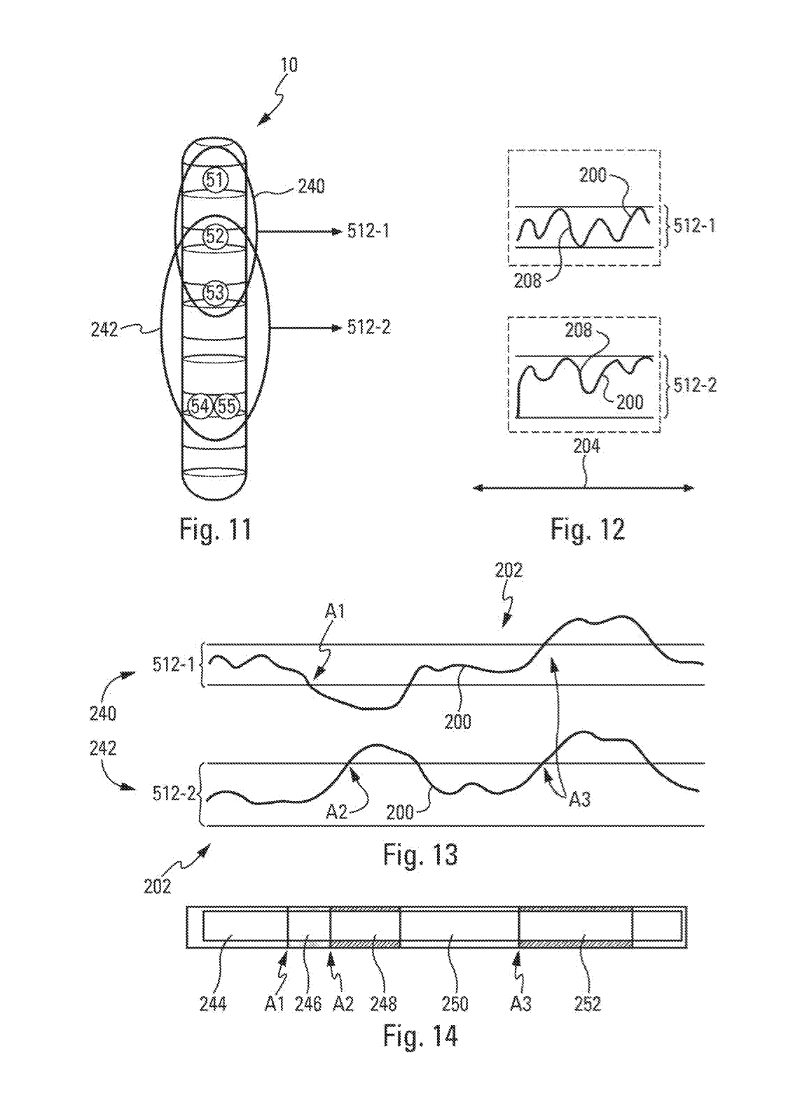

As illustrated in FIG. 11, the distillation unit 10 may be considered to comprise several zones 240, 242 each utilizing one said adjustment parameter. More particularly, each of the zones comprises separate concentration sensors or analyzers 51, 54, 55, some of said zones possibly having common concentration sensors or analyzers 52, 53. Provided here is a first zone 240 containing the first, second and third sensors 51, 52, 53 and a second zone 242 containing the second, third, fourth and fifth sensors 52, 53, 54, 55.

As illustrated in FIG. 12, one said nominal variation range 512-1, 512-2, to be used for each of said zones 240, 242 of the column 10 in the utilization phase, is determined.

As illustrated in FIGS. 13 and 14, one advantage of such a solution is to facilitate the identification of the faulty sensor(s).

In FIG. 13, it is seen that a first alert A1 is emitted that corresponds to the first zone 240 followed by a second alert A2 corresponding to the second zone 242, this second alert A2 coming after the first A1.

In FIG. 14, this is expressed by a first sequence 244 preceding the first alert A1, the first sequence corresponding to the emission of information of absence of failure. This first sequence 244 is followed by a second sequence 246 ranging from the first alert A1 to the second alert A2, during which second sequence information of probable failure is emitted. This second sequence 246 is then followed by a third sequence 248 occurring after the second alert A2, during which third sequence information of failure is emitted. Analysis of the series of sequences 244, 246, 248 makes it possible to believe that it is one of the sensors of the first zone 240 that is affected since the anomaly first appeared in this zone.

Referring again to FIG. 13, it is observed that, after a return to normal, a new alert A3 is emitted simultaneously for the two zones 240, 242.

In FIG. 14, said return to normal corresponds to the fourth sequence 250 and the new alert A3 corresponds to the start of a sixth sequence 252 in which information of failure is directly emitted, without passing through a step of emitting information of probable failure. This series of sequences 250, 252 makes it possible to believe that the anomaly affects the sensors 52, 53 common to the two zones 240, 242 since it appeared simultaneously therein.

The device for determining the concentrations in accordance with the invention mentioned above will of course be configured to enable such detections of failure. In this sense it comprises means for detecting the values 200 of said adjustment parameter departing from a nominal variation range of said parameter. Said means will be able to utilize, for this, a computer program executed, for example, by the processor 50 of said device. Said computer program is optionally stored on a recording medium.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1-15. (canceled)

16. A method for determining the concentrations of chemical components of a product in a distillation column, the method comprising the steps of:
implementing a model that estimates the concentration of the chemical components of the product based on measurements made by one or more sensors, said model having an adjustment parameter that utilizes operating variations of the column; and
detecting values of said adjustment parameter that depart from a nominal variation range of said adjustment parameter in order to diagnose a failure of one or more of said sensors.

17. The method as claimed in claim 16, comprising a prior step of establishing at least one said nominal variation range of said adjustment parameter.

18. The method as claimed in claim 17, wherein said prior step comprises a learning step, wherein all or some of the values taken by said adjustment parameter over at least one period of time, referred to as reference values, are recorded.

19. The method as claimed in claim 18, wherein said prior step further comprises a step of determining said nominal variation range from said reference values.

20. The method as claimed in claim 19, wherein said prior step further comprises a step of fusing data originating from several distillation columns and said step of determining said nominal variation range utilizes said data fusion to determine an overall nominal variation range.

21. The method as claimed in claim 16, further comprising a step of recording values taken by said adjustment parameter during an excursion of said adjustment parameter outside of said nominal variation range, referred to as the initial nominal variation range, and a step of modifying the initial nominal variation range to give an expanded range, taking into account values taken by the adjustment parameter outside of said initial nominal variation range, when a verification makes it possible to establish that the failure associated with said excursion does not exist.

22. The method as claimed in claim 16, wherein several nominal variation ranges are provided, each range corresponding to given operating conditions of said distillation column and said method comprises a step of determining the operating conditions of the distillation column and a step of selecting the corresponding nominal variation range.

23. The method as claimed in claim 16, wherein said distillation column comprises several zones each utilizing one said adjustment parameter, in which method one said nominal variation range is used for each of said zones.

24. The method as claimed in claim 16, wherein said model utilizes a propagation term in connection with a convection of said components along the column and an axial diffusion term in connection with a diffusion of said components in the column, the adjustment parameter making it possible to weight the effects of the diffusion with respect to the effects of the propagation.

25. The method as claimed in claim 16, additionally comprising the steps of:
measuring the concentration of at least one of said components in at least one location of the column; and
adjusting the model with the aid of the adjustment parameter determined from the concentration measured.

26. The method as claimed in claim 25, wherein:
the concentration of said chemical component is estimated at said location of the distillation column where the measurement took place with the aid of said model with a first value of said adjustment parameter,
an error is established between the estimated value and the measured value of the concentration,
a second value of the adjustment parameter is established as a function of said error,
the first value of the adjustment parameter is replaced by the second value in said model.

27. The method as claimed in claim 16, wherein the steps are carried out by a computer program comprising instructions for the implementation of the method, and the computer program is executed by a processor.

28. The method as claimed in claim 16, wherein the computer program is stored on a recording medium.

29. A device for determining the concentrations of chemical components of a product in a distillation column, said device comprising:
one or more sensors;
means for implementing a model that estimates the concentration of the chemical components, from measurements made by the sensor(s), said model utilizing an adjustment parameter that takes into account operating variations of the distillation column; and
means for detecting values of said adjustment parameter that depart from a nominal variation range of said parameter in order to diagnose a failure of one or more of said sensors.

30. The device as claimed in claim 29, wherein the distillation column is part of an air separation unit.

31. The device as claimed in claim 29, wherein the means for implementing the model is carried out by a computer program comprising instructions for the implementation of the method, and the computer program is executed by a processor.

32. The device as claimed in claim 29, wherein the means for detecting values of said adjustment parameter is carried out by a computer program comprising instructions for the implementation of the method, and the computer program is executed by a processor.
